(19) United States
(12) Patent Application Publication
(10) Pub. No.: US 2005/0228307 A1
(43) Pub. Date: Oct. 13, 2005

(54) Removable medical device adapter

(76) Inventor: Steve Gibree, Foster, RI (US)

Correspondence Address: IP STRATEGIES, 12 1/2 WALL STREET, SUITE I, ASHEVILLE, NC, 28801, US

(21) Appl. No.: 11/105,161

(22) Filed: Apr. 13, 2005

Related U.S. Application Data
(60) Provisional application No. 60/561,959, filed on Apr. 13, 2004.

(51) Int. Cl. A61B 5/00

(52) U.S. Cl. 600/549

(57) ABSTRACT

An adaptable detection component of a medical measuring device includes a first portion that detects a parameter, and a second portion that is removably attached to the first portion and which does not interfere with the parameter detection. The second portion is adapted to conform to an anatomical portion of a subject of a measurement using the device. The detection component can be formed integrally with the medical measuring device, or it can be adapted to be removably attached to the medical measuring device.

### REFERENCE TO RELATED APPLICATION

This is related to, and claims priority from, U.S. Provisional Patent Application No. 60/561,959, which was filed on Apr. 13, 2004.

### FIELD OF THE INVENTION

The invention relates generally to medical devices, and particularly to adapters for medical devices that can be used to adapt the medical device to suit the relevant physical dimensions of the patient on whom the device is used.

### BACKGROUND OF THE INVENTION

Many devices are made for use by people without consideration of the variations in size and proportion of different people. Sometimes, these differences are considered, but manufacturing efficiency dictates that device dimensions be tailored to the broadest range of potential users. Despite even the best measures taken by manufacturers, certain products will have dimensions and proportions that are not the best fit for at least some percentage of users.

This ergonomic shortcoming is particularly meaningful with respect to medical devices. Many medical devices that are used to take measurements for diagnostic purposes are manufactured in standard sizes that are more or less suitable for a large percentage of potential users, but which do not provide the best match for a large number of people. For example, the probe end of an ear thermometer, such as an infrared ear thermometer, is generally molded integrally with the rest of the instrument, and therefore is manufactured in a size and shape that is intended to conform to most ears. However, the size and shape of an infant's ear can vary a large amount from that of a full-grown adult. Even among an adult population, the variance in ear depth, ear opening size, and internal ear canal shape can vary to an extent that measurements taken with a standard probe might be inaccurate, and the action of taking the measurement can be uncomfortable or even painful.

### BRIEF DESCRIPTION OF THE INVENTION

According to an aspect of the invention, an adaptable detection component of a medical measuring device includes a first portion that detects a parameter, and a second portion that is removably attached to the first portion and which does not interfere with the parameter detection. The second portion is adapted to conform to an anatomical portion of a subject of a measurement using the device. The detection component can be formed integrally with the medical measuring device, or it can be adapted to be removably attached to the medical measuring device.

More particularly, the first portion can perform detection functionality for the medical measuring device. The first portion can be an inner portion, and the second portion can be disposed outside of the first portion.

According to an exemplary embodiment, the medical measuring device is a thermometer, for example, an ear thermometer, such as an infrared thermometer. In this case, the second portion can be an earpiece. The first portion can be a probe that detects a temperature proximate to the probe, and the second portion can be a sleeve that fits over the probe. The sleeve can include an aperture that provides access to an end of the probe through the sleeve.

The second portion can be removeably attached to the first portion in any of a number of ways. For example, the second portion can be friction fit to the first portion. Alternatively, either portion can include raised elements that engage recessed elements of the other portion to hold the second portion in place, or both portions can be threaded such that they engage each other. As another alternative, one portion can include a raised annular ridge that engages an annular depression on the other portion to hold the second portion in place.

In general, the second portion can be adapted to conform to an anatomical portion of a subject human or animal. Also, the component can be provided as part of a set of components, each of which has a size and/or shape that is different than the size/shape of another component in the set.

### BRIEF DESCRIPTION OF THE DRAWINGS

FIG. 1 is a block diagram of a medical measuring device having a removable adapter.

FIG. 2a is a first view of an exemplary embodiment in which an ear thermometer has a removable adapter.

FIG. 2b is another view of the exemplary embodiment of FIG. 2a.

FIG. 2c is a close-up view of detector and adapter portions of the exemplary embodiment of FIG. 2c.

### DETAILED DESCRIPTION OF THE INVENTION

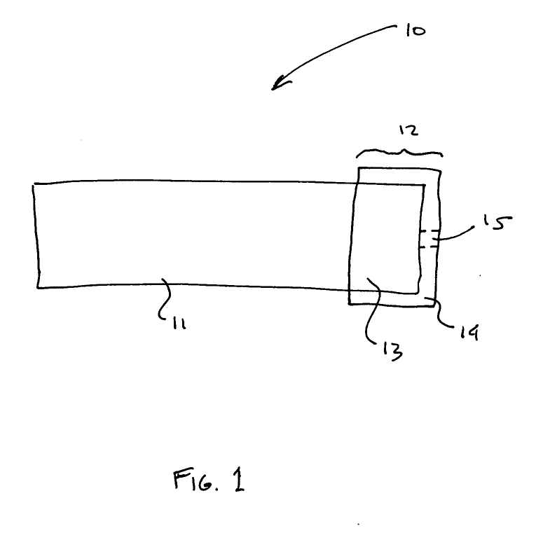

As shown in FIG. 1, a medical measuring device 10 includes a device base 11 and a detection component 12, and is used to detect and measure the presence of a parameter for a medical purpose, such as diagnostics. The detection component 12 includes a detector portion 13 that detects a parameter, and an adapter portion 14 that is removably attached to the detector portion 13 and which does not interfere with the parameter detection. For example, the adapter portion 14 can be made from a material that is transparent to the parameter being detected, or to a signal generated by the detector portion 13 in order to stimulate or detect the parameter. Alternatively, the adapter portion 14 can include an aperture 15 through which the parameter is detected.

The adapter portion 14 is adapted to conform to or accommodate an anatomical portion of a subject of a measurement using the device 10. The detection component 12 can be formed integrally with the medical measuring device 10, or it can be adapted to be removably attached to the medical measuring device 10.

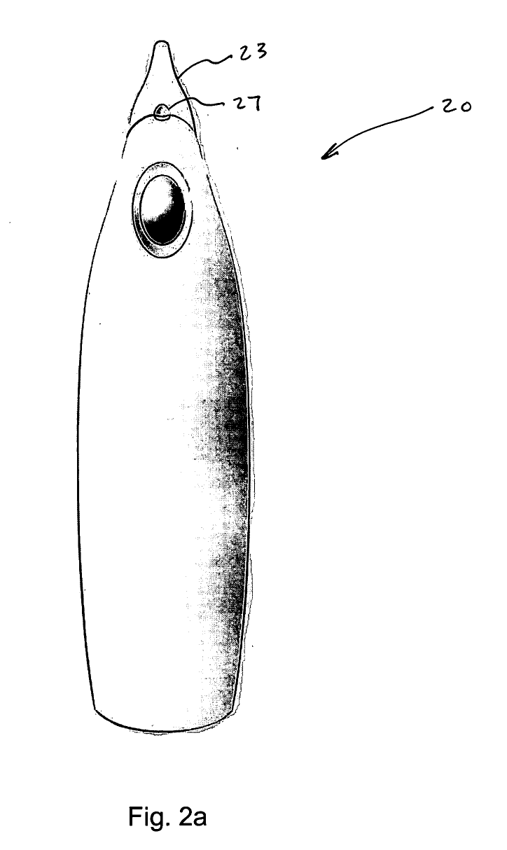

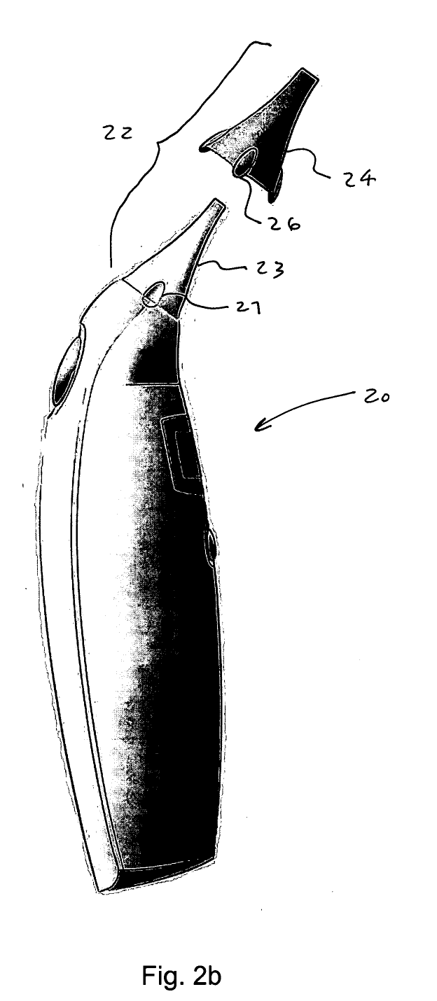

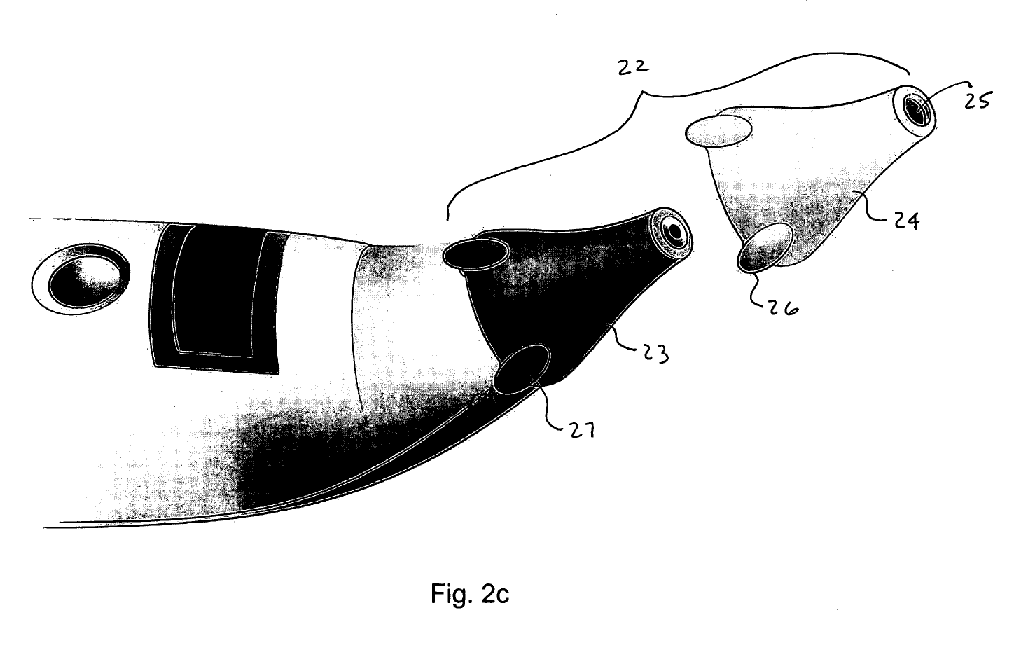

As shown in FIGS. 2a and 2b, an exemplary embodiment of the medical measuring device is a thermometer, for example, an ear thermometer, such as an infrared thermometer, which includes a temperature detection component 22. In this embodiment, the adapter portion is an earpiece 24. The detector portion is a probe 23 that detects a temperature proximate to the probe 23, and the adapter portion is a sleeve 24 that fits over the probe 23. As shown in more detail in FIG. 2c, the sleeve 24 can include an aperture 25 that provides access to an end of the probe 23 through the sleeve 24.

The adapter portion 14 can be removeably attached to the detector portion 13 in any of a number of ways. For example, the adapter portion 14 can be friction fit to the detector portion 13, or both portions can be threaded. Alternatively, one portion can include a raised annular ridge that engages an annular depression on the other portion to hold the adapter portion 14 in place. As another alternative, as shown in the exemplary embodiment, the sleeve 24 can include raised elements 26 that engage recessed elements 27 of the probe 23 to hold the sleeve 24 in place.

In general, the adapter portion 14 is adapted to conform to or accommodate an anatomical portion of a subject human or animal. In the exemplary embodiment, the sleeve 24 is adapted to conform to a patient's ear, at least so as to position the probe 23 properly for taking an accurate body temperature measurement. In order to adapt to different patients having ears of different sizes, shapes, and proportions, the sleeve 24 can be provided as part of a set of sleeves, each of which has a different size and/or shape than the size/shape of another component in the set.

Thus, the adapter portion 14 is shaped to provide substantially the same parameter-measuring qualities as the detector portion 13, but provides a better fit for a particular user or class of users. In the exemplary embodiment, a thermometer might be molded with a probe 23 that has a size and shape that is suitable for most adults, but which might be too large for an infant. A selected removable sleeve 24 fits over the probe 23, and terminates in a smaller, narrower tip that is more suitable for use with an infant. Alternatively, the external shape of the removable sleeve 24 can be wider than that of the probe 23, to restrict the depth to which the probe can be inserted. It is contemplated that removable adapter portions 14 of various shapes and sizes can be provided, depending on the user and the device, and the present invention is not restricted in scope to any particular size or shape.

Particular exemplary embodiments of the present invention have been described in detail. These exemplary embodiments are illustrative of the inventive concept recited in the appended claims, and are not limiting of the scope or spirit of the present invention as contemplated by the inventor.

1. An adaptable detection component of a medical measuring device, comprising:
a first portion that detects a parameter; and
a second portion that is removably attached to the first portion and which does not interfere with the parameter detection;
wherein the second portion is adapted to conform to an anatomical portion of a subject of a measurement using the device.

2. The component of claim 1, wherein the component is formed integrally with the medical measuring device.

3. The component of claim 1, wherein the component is adapted to be removably attached to the medical measuring device.

4. The component of claim 1, wherein the first portion performs detection functionality for the medical measuring device.

5. The component of claim 4, wherein the first portion is an inner portion, and the second portion is disposed outside of the first portion.

6. The component of claim 1, wherein the medical measuring device is a thermometer.

7. The component of claim 6, wherein the thermometer is an ear thermometer.

8. The component of claim 6, wherein the thermometer is an infrared thermometer.

9. The component of claim 7, wherein the second portion is an earpiece.

10. The component of claim 7, wherein the first portion is a probe that detects a temperature proximate to the probe.

11. The component of claim 10, wherein the second portion is a sleeve that fits over the probe.

12. The component of claim 11, wherein the sleeve includes an aperture that provides access to an end of the probe through the sleeve.

13. The component of claim 1, wherein the second portion is friction fit to the first portion.

14. The component of claim 1, wherein one of the first portion and the second portion includes raised elements that engage recessed elements of the other of the first portion and the second portion to hold the second portion in place with respect to the first portion.

15. The component of claim 1, wherein one of the first portion and the second portion includes a raised annular ridge that engages an annular depression on the other of the first portion and the second portion to hold the second portion in place with respect to the first portion.

16. The component of claim 1, wherein the first portion and the second portion include respective threaded surfaces that engage each other to hold the second portion in place with respect to the first portion.

17. The component of claim 1, wherein the second portion is adapted to conform to an anatomical portion of a subject human.

18. The component of claim 1, wherein the second portion is adapted to conform to an anatomical portion of a subject animal.

19. The component of claim 1, included as part of a plurality of components, wherein each said component has at least one of a size and a shape that is different than at least one of a size and a shape of another said component.

20. An ear thermometer, including the adaptable detection component of claim 1.
